(19) United States
(12) Patent Application Publication
(10) Pub. No.: US 2008/0254480 A1
(43) Pub. Date: Oct. 16, 2008

(54) MICROCYTOXICITY ASSAY BY PRE-LABELING TARGET CELLS

(75) Inventor: Nikola L. VUJANOVIC, Pittsburgh, PA (US)

Correspondence Address: FOLEY AND LARDNER LLP;SUITE 500, 3000 K STREET NW, WASHINGTON, DC, 20007, US

(73) Assignee: University of Pittsburgh - Of the Commonwealth System of Higher Education

(21) Appl. No.: 11/733,863

(22) Filed: Apr. 11, 2007

(51) Int. Cl. G01N 33/53

(52) U.S. Cl. 435/7.1

(57) ABSTRACT

The standard or original microcytotoxicity assay (OMCA) has significant advantages over other cytotoxicity assays, since it is able to detect both cell necrosis and apoptosis and it is simpler, safer, more practical, and more economical. OMCA has serious weaknesses, however, such as low accuracy, low selectivity, and low sensitivity. These drawbacks are ameliorated or eliminated by pre-labeling of target cell nuclei, for instance, with 5-bromo2′-deoxyuridine. This improved microcytotoxicity assay (IMCA) is readily adapted to a wide range of applications, such as screening of cytotoxicity drug candidates, selecting an anticancer cytotoxic therapy, detecting abnormalities including reduced tumor cell killing ability of NK cells in cancer patients, predicting outcome of cytokine therapy and immunotherapy, determining effectiveness of cytokine therapy and immunotherapy in follow up studies following treatment, determining effectiveness of anticancer cytotoxic therapy during and following therapy and ascertaining cytotoxic T cell activity during anticancer vaccination therapy.

### BACKGROUND OF THE INVENTION

The present invention relates to a so-called “microcytotoxicity assay” (MCA) that is improved, relative to conventional assays of this type, in terms of its consistency, sensitivity, and ease of implementation. In this description, citations to the literature, tabulated below, appear in parentheses.

Cytotoxicity assays are widely used to assess in vitro effectiveness of new cytotoxic agents, susceptibility of cells to cytotoxic drugs or immune effector mechanisms, and to measure antiviral or anticancer host immune resistance. To these ends, a wide variety of cytotoxicity assays has been developed and applied(1-9).

In general, conventional assays are based on relatively complex and laborious procedures, and they are not easy to perform. They utilize radioactive or toxic reagents; hence, their use often poses safety issues. In addition, the assays employ relatively large quantities of expensive reagents and tissue culture media as well as expensive equipments. Thus, they are not economical or practical.

Because of these disadvantages, existing cytotoxicity assays have not been standardized and applied in routine pharmacological or clinical studies. Consequently, there is an urgent need for a safe, economical, and readily implemental cytotoxicity assay, which can be standardized and adjusted for use in routine laboratory testing.

The microcytotoxicity assay has many of the advantageous characteristics over other cytotoxicity assays (4). It is simpler and easier to perform, and it does not utilize radioactive or toxic reagents, expensive reagents, expensive laboratory wares, or expensive equipment. The original microcytotoxicity assay consumes ten to fifty times less tissue culture media, target cells, immune effector cells or reagents and is suitable for routine assessment of cell-mediated cytotoxicity in patients, including those with low white blood cell counts, such as patients who suffer from acquired immunodeficiency syndrome (AIDS) or have undergone a cytotoxic treatment.

Thus, MCA is simpler and safer as well as more economical and practical than other cytotoxicity assays in use. In addition, MCA detects both cell necrosis and apoptosis. Therefore, MCA might have a broad application, therefore, but it has several serious weaknesses.

For instance, the original MCA (OMCA) is based on optical analysis of May Grunwald-Giemsa (MGG) stained whole tumor cells, which are variable not only in size and shape but also in quality and intensity of staining. Additionally, nuclei and cytoplasm of the target cells are not consistently and distinctly stained and, because target cells are often in contact each other, they could be mistaken for single objects. In OMCA, therefore, target cells are not always morphologically distinguishable as individual and uniformed objects, and can not be accurately counted, particularly using computerized counting system. Furthermore, in cell-mediated cytotoxicity assay, variable numbers of immune effector cells can remain in microwells of plates at the end of assay, thereby contributing significantly to the counting errors.

### SUMMARY OF THE INVENTION

To address these and other shortcomings in conventional cytotoxicity assay technology, the present invention provides, in accordance with its aspects, an improved microcytotoxicity assay (IMCA) comprised of: (A) providing a culture of target cells adhered to a surface, wherein the target cells are exposed to a labeling agent that labels nuclei of the target cells; then (B) treating the target cells with a putative cytotoxic agent; and thereafter (C) assessing the number of the target cells that remain adhered to the surface. In a preferred embodiment, the surface in question is a surface of a microwell into which the target cells are plated. Thus, the invention contemplates an assay where the target cells are distributed among a plurality of microwells.

In accordance with another aspect, the present invention provides a combination that is comprised of (i) a suspension of cells that can form an adherent layer, wherein the target cells are labeled by an agent that labels nuclei thereof, and (ii) instructions for the use of the cells to effect a microcytotoxicity assay as described above. The combination also can comprise (iii) a microcytotoxicity plate, wherein the aforementioned instructions relate the use to the plate for effecting the microcytotoxicity assay.

### BRIEF DESCRIPTION OF THE DRAWINGS

FIG. 1. Photographic rendition of morphology of MGG-stained or 5-bromo-2′-deoxyuridine (BrdU)-labeled target cells in microwells of Tarasaki plates: cytotoxic effects of the immune effector cells (peripheral blood mononuclear leukocyte, PBMNL) or UV irradiation in OMCA and the improved microcytotoxicity assay (IMCA) of the present invention, respectively. OMCA: MGG-stained BT-20 tumor target cells, (A) untreated and (B) following treatment with PBMNL (24 hours, 200:1=E:T ratio). In (B), note the visible remains of PBMNL. BrdU-labeled BT-20 cells, (C) stained with non-reactive isotype matched control or (D) anti-BrdU mAbs. (E and F) Morphological changes of BrdU-pre-labeled BT-20 cells 24 hours after UV irradiation. IMCA: BrdU-pre-labeled BT-20 cells, (G) untreated or (H) after treatment with PBMNL (24 hours, 200:1=E:T ratio).

FIG. 2. Graph depicting BrdU concentration versus incubation period for labeling of target cells in microwells of Tarasaki plates. BT-20 tumor cells (250/microwell) were seeded in the miniplates and incubated for 24 hours. Various concentrations of BrdU then were added to the microwells containing adherent tumor cells, and the incubation was continued for various periods of time, as indicated. After this labeling, the adherent tumor cells were fixed and stained, with an isotype-matched control or an anti-BrdU mAb, and then visually analyzed. Each point on the figure represents a mean value of a triplicate. SD was less than 10% of the mean values.

FIG. 3. Graph illustrating kinetics of PBMNL-mediated killing of tumor cell targets, determined in OMCA and in IMCA. BT-20 tumor cells (250/microwell) were seeded in Tarasaki plates in the absence or presence of 5 μM of BrdU and incubated for 24 h. The unlabeled and BrdU-labeled target cells were then exposed to PBMNL in OMCA and IMCA, respectively, for the indicated periods of time. Following this incubation, non-adherent cells were removed and the assays were completed, as described in details below. The assays were performed in six replicates and four different effector:target (E:T) ratios (i.e., 200:1, 100:1, 50:1 and 25:1). The results are LU₂₀/10⁷ NK cells. The differences between the results obtained in IMCA and OMCA were significant (p<0.005).

### DETAILED DESCRIPTION OF PREFERRED EMBODIMENTS

The present inventor has discovered that the serious weaknesses of OMCA, discussed above, are largely overcome by selective pre-labeling of target cell DNA, as can be effected, for example, with 5-bromo-2′-deoxyuridine (BrdU), a DNA metabolic label. The incorporated label in nuclear DNA of target cells can be detected selectively at the end of IMCA. With BrdU as label, detection can be by means of specific immunocytochemical staining with peroxidase- or alkaline phosphatase-conjugated anti-BrdU antibodies (11).

Pre-labeling of target cells, pursuant to the present invention, has not been previously used in MCA. It allows for selective and distinct staining of target cell nuclei, which are morphologically much more uniform objects in size and shape than whole target cells, and, being separated by unstained cytoplasm of neighboring cells, are better defined individual objects for counting than whole cells. In accordance with the present invention, therefore, pre-labeling of target cells before cell-mediated cytotoxicity excludes counting errors of counting remaining immune effector cells, which otherwise would be counted as target cells.

In addition, selective pre-labeling of target cell DNA and specific staining of cell nuclei, in accordance with the invention, enables an improved analysis of specific morphological changes in nuclei during apoptosis.

Furthermore, the improved microcytotoxicity assay (IMCA) of the present invention is readily implemented in microwells, which requires minute quantities of reagents and, hence, is highly economical.

### General Principles of the Assays

The conventional MCA is performed using adherent cell targets (4). Target cells are suspended, plated in microwells of Tarasaki tissue culture mini-test plates and incubated overnight to adhere to the plastic surface. Following the adherence, target cells are treated with cytotoxic agents (drugs, radiation, cytotoxic immune cells). Dead cells become non-adherent during the cytotoxic treatment, and they are removed by washing the wells. Remaining adherent (viable) target cells are fixed, stained, and optically counted. Target cells treated with cytotoxic agents were compared with those untreated.

While operationally similar to OMCA, as further discussed below, the methodology of the present invention entails the use of target cells that are pre-labeled, preferably in their nuclei, before a putative cytotoxic treatment. After cytotoxic treatment, removal of dead cells and fixation of remaining viable cells, selective staining of incorporated label in target cell nuclei, and optical counting of labeled nuclei are effectuated.

In this description, an exemplified embodiment of the present invention involves the use of BrdU, for the aforementioned pre-labeling, and detection of the labeling by (a) specific immunochemical staining and (b) the use of light microscopy-based instrumentation. In principle, the incorporated BrdU in cell nucleus DNA could be visualized as well through the use of fluorochrome-conjugated antibodies.

Moreover, other nuclear markers or stains for in vivo use could be employed, such as 4′,6-diamidino-2-phenylindole (DAPI), one of the Hoechst fluorescent stains, 33258 and 33342, or one of the cell-permeant cyanine nucleic acid stains that is marketed, under the SYTO mark, by Invitrogen Corporation (Carlsbad, Calif.). The present invention also contemplates an assay that entails the pre-labeling of target cells by means of a fluorescent cell tracker of viable cells, as described above, in combination with one or more fluorescent probes for apoptosis or necrosis, such as, respectively, Annexin V or caspase-3 and propidium iodide or 7-amino-actinomycin D (7-AAD).

After fluorochrome labeling, the results of the assay can be analyzed visually via fluorescent-microscopy observation or by a semi-automated procedure, using a computerized cytometry fluorescence system (13), such as one incorporating the ArrayScan® reader, product of Cellomics, Inc. (Pittsburgh, Pa.). The illustration of the invention described below involved the use of Tarasaki 60-well microcytotoxicity plates, but other multi-well plate formats may be employed, such as a 384-well, flat-bottom plate, which is well-suited to the use of fluorochrome tags.

### Target Cells

Target cells in an microcytotoxicity assay comprise normal cells and tumor cells that are adherent to a surface. In this regard the phrase “tumor cell” denotes a cell, which can be malignant or benign, that are characterized by spontaneous proliferation. By contrast, “normal cells” are cells that proliferate in the presence of growth stimulatory factors.

For an illustrative implementation of the inventive technology, several adherent tumor cell lines were used, obtained from tumor tissues of cancer patients, including CRL-1620 glioma, MTB-72 melanoma, HTB-47 renal cell carcinoma, HTB-178 lung adenocarcinoma, BT-20, MCF-7, SK-BR-3 and HTB-126 breast carcinomas (ATCC, Rockville, Md.), and PCI-13 squamose cell carcinoma of head and neck (SCCHN, UPCI, Pittsburgh, Pa.). The cell lines were grown under standard cell culture conditions, as previously described (6). They were cultured as adherent monolayers and utilized in experiments when they reached 75% of confluency. Cell suspensions were prepared from the monolayers by mild trypsinization, using 0.05%/0.53 mM trypsin/EDTA in Hanks balanced salt solution (HBSS, GIBCO BRL, Life Technologies, Long Island, N.Y.) and 2-minute incubation at 37° C., followed by one wash in ice-cold RPMI-1640 medium supplemented with 10% FCS (GIBCO BRL) (RPMI-10) and a trypsin inhibitor (Cell Systems, Kirkland, Wash.). Then, the cell suspensions were washed two more times in RPMI-10 alone. After this washing, target cells were counted, diluted, to deliver between 100 and 500 cells in 10 μl, and plated into the microwells of Tarasaki plates.

In addition to the above-mentioned tumor cell lines, target cells in IMCA can be other adherent tumor or normal cell lines, as well as freshly isolated, shortly cultured tumor cells from cancer patients' tumors. Furthermore, leukemia cell lines and freshly isolated leukemia cells from leukemia patient blood, and normal non-activated and activated PBMNL and bone marrow nucleated cells, which grow as non-adherent cell suspensions, likewise can be used in an assay of the present invention, after the cells are anchored, using a cell-adhesive solution of poly-L-lysine, to a planar plastic surface of a well in a microcytotoxicity plate.

### Cytotoxic Agent

The term “cytotoxic agent” as used herein refers to any agent that has a toxic effect on cells. Cytotoxic agent includes, but is not limited to, chemical compounds or small molecules; radiation, e.g. UV radiation and X-ray radiation; and immune effector cells. One skilled in the art would understand that immune cells are white blood cells or leukocytes produced in the bone marrow and thymus that form the immune system (PBMNL and lymphoid tissues) and defend the body against infectious disease, cancer and foreign materials. Immune effector cells are cytotoxic cells that include natural killer (NK) cells, cytotoxic T lymphocytes (CTLs), and natural killer-T (NK-T) cells and dendritic cells (DC) and kill other cells such as tumor cells and virally infected cells. PBMNL are representative part of the immune system containing all immune cells including immune effector cells and are most commonly used to examine the immune system in human. PBMNL are obtained from heparinized blood by centrifugation on Ficoll-Hypaque density gradient.

### UV Irradiation of Tumor Cells

To induce apoptosis, adherent tumor cells in microwells of Tarasaki plates were washed twice with PBS and exposed to the germicidal lamp in the laminar flow hood, which emitted radiation in the UVC region with a peak of 254 nm wavelength. The incident-dose rate at the level of the plate was 1.2 W/m² as determined by a Black-Ray UV meter (Model J225; American UV Co., Chatham, N.J.). Tumor cells were irradiated with a total dose of 610 J/m².

### MCA

As used herein, the term “microcytotoxicity assay (MCA)” means, in general, an assay in a micro format that detects cell destruction caused by a cytotoxic agent. The term “original microcytotoxicity assay (OMCA)” refers to an MCA that is performed according to a conventional, standard procedure before this invention. By contrast, the term “improved microcytotoxicity assay (IMCA)” refers to an MCA that is performed according to the present invention, i.e. with an inventive step of selectively pre-labeling of target cells prior to the treatment with a putative cytotoxic agent.

In relation to the OMCA, as described, for example, by Takasugi and Klein (4), the assay implemented for this illustration of the invention was modified as follows: The assay was performed in Tarasaki 60-well minitest culture plates (Nalge Nunc International, Denmark). Target cells were suspended in culture medium and seeded into the microwells (100-500 cells/10 μl/well), using pipetor P20 or 250-μl 6-channel Hamilton syringe (Hamilton, Whitter, Calif.). The cells were cultured at 37° C. in an atmosphere of 5% CO₂ and absolute humidity for 24 hours, to allow their adherence. Various numbers of immune effector cells then were added to the target cells (5 μl/well), to obtain appropriate effector:target (E:T) cell ratios. Each E:T ratio was assessed in six replicates. Six to 18 control wells per plate were supplemented with 5 μl of tissue culture medium alone. The plates were further incubated for 24 hours. In some experiments, to determine optimal duration of cytotoxicity, kinetic studies were performed by co-incubating target and immune effector cells between 4 and 48 hours. The test was completed by upside-down positioning of plates for 30 minutes, following by six washes of microwells with warmed (37° C.) RPMI 1640 medium, to eliminate dead (non-adherent) and preserve viable (firmly adherent) cells. The washing was performed by adding 10 ml of RPMI 1640 medium to each Tarasaki plate, gently waving of the washing medium in the plate, and pouring out the washing medium by inverting of the plate.

Viable, adherent cells were fixed for 10 minutes in absolute methanol and were stained with May-Grunwald Giemsa. The test was scored under microscope, by both ocular and computerized, semi-automated optical techniques of counting of the number of remaining cells in the wells. Percentages of cytotoxicity were determined using the following formula:

$$\frac{\begin{matrix}( Number of cells in control wells ) - \\ ( Number of cells in experimental wells )\end{matrix}}{N umber of cells in control wells} \times 100$$

Human tumor cell lines used in this illustration of the invention were slowly proliferating cells with a doubling time of about 60 to 72 hours. Therefore, the number of target cells remained relatively constant during the assay period between 24 and 48 hours.

### IMCA

As noted, the main difference between the two assays contrasted in this illustrative study was that in IMCA, but not in OMCA, target cells were pre-labeled with BrdU. BrdU labeling of cell nuclear DNA has been in use for many years as an excellent method for detection of proliferating cells both in vitro and in vivo (10-12). An ELISA specific for BrdU has been developed for detection of soluble DNA fragments in the supernatant of BrdU-pre-labeled target cells undergoing apoptosis (see catalogue of Boehringer Mannheim Co).

BrdU-labeling of cells is based on the ability of BrdU to be incorporated into cell nuclear DNA in place of thymidine, during the S phase of cell cycle. Labeling of cell nuclear DNA by BrdU is a simple, efficient and highly reproducible procedure. For the in vitro labeling, Boehringer Mannheim recommends that soluble BrdU (10 μmol) is added to culture medium that contain cells, growing on cover slips or in tissue culture chamber slides, and is co-incubated with the cells for 30 minutes. Cell nuclei that have incorporated BrdU into DNA then are detected, using BrdU-specific antibodies and enzyme- or fluorochrome-conjugated secondary antibodies.

In the present study, the reagents used for this purpose were from the BrdU-labeling and detection kit II (Catalog No. 1299 964, Boehringer Mannheim). The basic technical principles for BrdU-labeling of cell nuclear DNA were similar to those described in the Boehringer Mannheim procedure. To accommodate its new application in IMCA, however, the original labeling procedure was modified significantly, as described in the next section.

For purposes of implementing the present invention, one would turn to the combination of (i) a suspension of cells that can form an adherent layer, as discussed above, where the cells are labeled by BrdU or another agent that labels nuclei, with (ii) instructions for the use of those cells to effect a microcytotoxicity assay of the invention. Thus, a user of IMCA could receive, in kit format, a receptacle containing pre-labeled, frozen tumor cell targets in suspension, plus a written protocol for the assay. Alternatively, such a kit could include cytotoxicity plates with adherent, pre-labeled target cells ready to use in the assay, as well as the instructions for the assay. As noted, the kit could include tumor cell targets, such as leukemia cells, that grow as non-adherent cell suspension. Utilizing non-adherent tumor cells in IMCA, according to the invention, would entail anchoring them to plastic microwell surfaces in the assay plates, using the cell-adhesive solution poly-L-lysine. Accordingly, a kit of the invention could include a receptacle containing such a cell-adhesive solution.

BrdU-labeling of target cells in microwells. Suspensions of tumor target cells were prepared from their adherent monolayers, as described above. Target cells were resuspended in tissue culture medium (e.g., RPMI-1640 supplemented with 10% FCS) in an adequate concentration, to contain 100 to 500 cells per 10 μl. BrdU solution was added to this cell suspension, to get its final concentration of 5 μM. In some experiments, to determine the optimal labeling conditions, the concentration of BrdU was varied. The cell suspension was distributed in a volume of 10 μl per each microwell of Tarasaki plates and incubated at 37° C. in an atmosphere of 5% CO₂ and 100% humidity for 24 hours, both to incorporate BrdU into the nuclear DNA of target cells and to allow adherence of the cells. Following this pre-incubation, target cells were washed. That was effected first by inverting the plates and applying a brisk shake, to remove from the wells the culture medium containing unutilized BrdU. Then the washing of cells in the plates was performed three times, by adding and removing of warm (37° C.) medium.

Induction of target cell killing. After the washing of target cells, the cytotoxicity assay was performed. In a cell-mediated cytotoxicity assay, immune effector cells were resuspended in RPMI-10 and added in a volume of 10 μl to the each well containing BrdU-labeled target cells. In control wells, medium alone was added in the same volume. In UV irradiation-induced cell death, target cells were washed twice with ice-cold PBS and directly exposed to UV light, as described above. Control wells contained untreated cells. After UV-irradiation, PBS was removed and target cells were washed and supplemented with 10 μl/well of cell culture medium. Following these treatments, the cells were incubated at 37° C. in an atmosphere of 5% CO₂ and 100% humidity for 24 hours. To determine the optimal conditions, in some experiments, the time period of incubation was varied from 4 to 48 hours.

Detection of BrdU incorporated in the cell nuclear DNA. Following the induction of target cell killing, dead (non-adherent) cells and/or immune effector cells were washed off, as described for MCA. After this washing, an additional gentle washing was applied with ice-cold PBS. In some experiments, to preserve dead cells in microwells for morphological examination, the washing was performed carefully, without inverting plates, by slowly adding to and removing from the plate 10 ml of RPMI 1640 medium using a pipet. The remaining cells were fixed at −20° C. in 10 ml per plate of 70% ethanol solution in 50 mM glycine buffer, pH 2.0, for 45 minutes.

The wells then were washed three times with the washing buffer from the kit. To stain BrdU incorporated into the cell nuclei, the cells were covered with 4 μl per microwell of appropriately diluted (e.g., 5 μg/ml) mouse anti-BrdU monoclonal antibody (mAb) or isotype matched (IgG1) non-reactive control mAb and incubated at 37° C. in an atmosphere of 100% humidity for 30 minutes.

The wells were washed three times with the washing buffer. The cells then were covered with 4 μl per microwell of goat anti-mouse Ig antibodies, conjugated with alkaline phosphatase (AP) (diluted 1:10), and were incubated as described for the primary antibody. The plates were washed 3 times with the washing buffer. The cells were covered with 4 μl per microwell of the freshly prepared color-substrate solution and incubated for 15 minutes at room temperature. The color reaction was stopped by a washing with PBS. The cells were covered with 4 μl per microwell of crystal/mount mounting medium (Biomeda Co., Foster City, Calif.).

Analysis of MCA. Eye-based (visual) and/or computerized, semi-automated optical cell counting were performed. Darkly and homogeneously stained, regular contours, oval shape nuclei of intact target cells were scored in both control and experimental wells. Visual counting was done using an inverted microscope (Olympus, Japan) and a magnification ×200. Computerized optical counting was performed on a Axoplan 2 research microscope for transmitted light bright field, using a custom designed counting program using the KS-400 image analysis software system (Carl Zeiss, Inc., Thornwood, N.Y.).

Each microwell with cells (cell nuclei) to be counted was placed under a microscope video camera. The images of cells (cell nuclei) in the microwells were scanned, corrected for a background color, obtained with cells (cell nuclei) stained with the isotype-matched control mAb, enhanced in sharpness and contrast and smoothed. The cell nucleus images was converted to binary images (white cell nuclei on a black background). Another image manipulation was used to better separate close neighboring tumor cells, to be able to count them as individual objects. Finally, the selected objects were counted using a computer. In some experiments, a differential analysis of the intact cells and those undergoing apoptosis (DNA condensation, collapse of cell nuclear material, shrinkage of nuclei, irregularity of nuclear shape, and fragmentation of nuclei) was performed.

### Statistical Analysis

Statistical analyses of the results were performed using the Wilcoxon's signed-rank pair and Mann-Whitney U tests. Differences were considered significant when the P value was <0.05.

### Results

Most of cancer cells or mitogen-stimulated lymphocytes are dividing cells, potentially able to incorporate in their nuclear DNA a specific metabolic label, such as BrdU. In principle, therefore, it seemed possible that an entire cell population could be labeled with BrdU, under optimized conditions.

On the other hand, no one heretofore had attempted to apply BrdU labeling of cell nuclei as a marker of cell populations or to measure cell death in MCA; hence, the nature of “optimization” in this context was unclear. In addition, BrdU-based assays have been expensive when performed in relatively large tissue culture dishes, consuming large quantities of reagents.

Accordingly, the present inventor undertook a series of experiments to determine whether BrdU labeling of cell nuclei can be performed in miniculture conditions, efficiently and inexpensively, to tag cell populations and/or to measure cytotoxicity.

### BrdU Labeling of the Cell Nucleus DNA in Target Cells

To determine optimal conditions for labeling target cells with BrdU, the inventor tested suitability of various tissue culture dishes, necessity that target cells are in suspensions (non-adherent) or monolayers (adherent), various concentrations of BrdU and various time periods of co-incubation of target cells with BrdU. A microwell of Tarasaki plate has volume of 15 μl, and its bottom surface can be covered with only 1 μl of a reagent; this is 100 to 1,000 times smaller volume than that previously used to detect cell proliferation in chamber slides or on cover slips, respectively.

This fact indicated that BrdU-based assays might be possible to perform in microwells of Tarasaki plates using minute amounts of reagents and that thus modified assays might be very economical. Utilization of reagents in small volumes might result in their drying during an assay. Accordingly, the inventor first assessed resistance to drying of 1 to 15 μl of water-based solutions in microwells of Tarasaki plates kept at 37° C. in an atmosphere of 100% humidity for 1 to 72 hours.

By this approach, the inventor demonstrated that 1-4 or 10-15 μl of the solutions per a microwell persisted, unchanged under the applied conditions, during 2 and 72 hours, respectively. In further experiments, he determined that target cells could be labeled with similar efficiency in tissue culture flasks or wells of various sizes, including microwells of Tarasaki plates. He also showed that target cells either in suspensions or in monolayers had similar excellent abilities to incorporate BrdU.

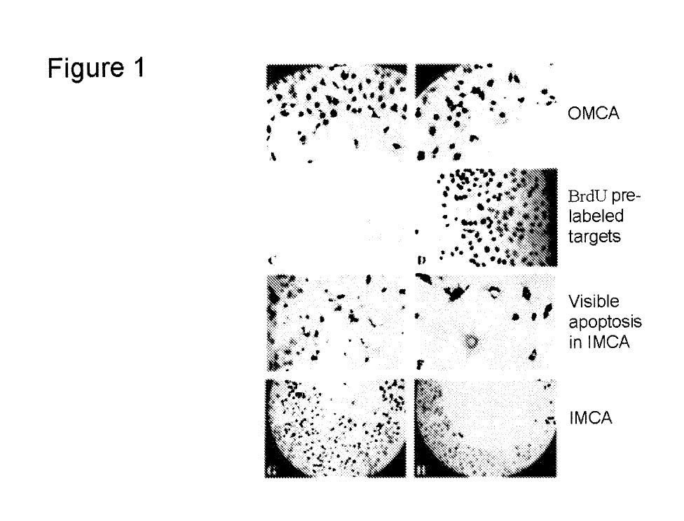

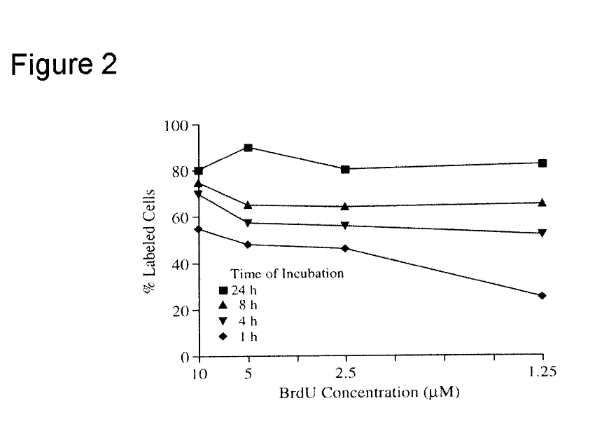

Furthermore, the inventor demonstrated, by incubating target cells for various period of time (from 1 to 24 h), in the presence of various concentrations of BrdU (from 1 to 10 μM), that optimal labeling of cell nuclear DNA (strong and uniformed labeling of 90% of nuclei) could be achieved if target cells were co-incubated with 5 μM of BrdU for 24 hours (FIGS. 1D and 2). Further incubation of thus labeled target cells for 24 to 48 hours in BrdU-free cell culture medium did not significantly influence the percentage of labeled target cells or intensity of the cell nuclear labeling (FIG. 1G). In further studies, therefore, target cells were labeled in microwells of Tarasaki plates during their 24-hour adherence to plastic surface in the presence of 10 μl/microwell of 5 μM of BrdU.

### Immunochemical Staining Of BrdU-Labeled Nuclei Of Target Cells in Microwells of Tarasaki Plates

Next, the inventor tested volumes and concentrations of the reagents as well as conditions of incubations necessary to obtain optimal immunocytochemical staining of BrdU incorporated into DNA of target cell nuclei. He determined that only 1 to 10 μI/microwell of the antibodies or enzyme substrate were necessary to apply to obtain consistently specific and distinct immunochemical staining of BrdU-labeled target cells in Tarasaki plates.

In the further studies, the inventor used 2 to 4 μl/microwell of the antibodies or enzyme substrate. These reagents were applied in similar concentrations to those suggested by Boehringer Mannheim. On the basis of data obtained in additional experiments, the inventor chose conditions as optimal for immunochemical staining of BrdU-labeled cell nuclei in microwells of Tarasaki plates. He also showed that not only composition of fixative (70% ethanol in 50 mM glycine buffer, pH 2.0) but also temperature (−20° C.) and time of incubation (45 minutes) during fixation of target cells were important for accurate immunostaining of BrdU-labeled target cells in microwells. All other reagents, their concentrations and conditions of utilization provided and/or suggested by Boehringer Mannheim were also found to be suitable for immunostaining of BrdU-labeled nuclei of tumor cells in Tarasaki plates.

### Preservation of BrdU-Labeled and Immunostained Target Cells

Following immunocytochemical staining of BrdU incorporated into cell nuclear DNA, the specifically stained nuclei were well defined for a few hours. After this period of time, cells were substantially dried out and staining of nuclei became less intensive and defined. To fully preserve the specific staining of cell nuclei for a prolonged period of time and to prepare cells for analysis, immediately after co-incubation of cells with color-substrate and their last PBS washing, the inventor added to each microwell with stained cells 4 μl of crystal/mount mounting media (Biomedia). This amount and type of mounting media were defined to both completely cover the bottom of microwells and provide optimal optical conditions for visual or computerized analysis of cell nuclei. In addition, cell nuclei thus treated were morphologically preserved for an accurate analysis for a couple of weeks.

### Morphology of BrdU-Labeled Target Cells

Microwells of Tarasaki plates have been used for morphological analysis of MGG- or crystal violet-stained adherent target cells in OMCA (4). These studies indicated that Tarasaki plates might be suitable for morphological studies of adherent cells when multiple replicates, multiple samples, and/or multiple experimental conditions are examined together and compared.

For the first time, therefore, the inventor assessed the utilization of Tarasaki plates for morphological analysis of pre-labeled adherent cells, according to the present invention. Target cells thus prepared consistently showed a staining that was specific, distinct, selective, strong and uniform in quality and intensity, as well as well defined nuclear morphology, uniform size and shape, and a clear segregation of neighboring cell nuclei (FIGS. 1C, D, G, H). Being separated by unstained cytoplasm of neighboring cells, BrdU-labeled nuclei appeared as well defined, easily countable, individual objects. Furthermore, morphological changes of nuclei during apoptosis were found distinct and well detectable following BrdU labeling (FIGS. 1E and F).

Thus, this procedure of the invention ideally prepared cell nuclei for optical counting. In contrast, whole tumor cells, stained with MGG or crystal violet, were variable not only in size and shape but also in quality and intensity of staining (FIGS. 1A and B). Additionally, nuclei and cytoplasm of cells so stained were not consistently and distinctly stained and morphologically defined. Often being in contact with each to other, groups of cells thus could be mistaken for single objects in counting. Accordingly, target cell nuclei that were BrdU-labeled, pursuant to the present invention, were much more consistent and better defined objects for optical counting than whole target cells stained with MGG or crystal violet.

### Functional Properties of BrdU Labeled Target Cells

In order to determine whether BrdU labeling of cell nuclear DNA affects cellular functions, the inventor examined viability (by trypan blue dye exclusion assay) and proliferation (by evaluation of increase of cell numbers in culture) of target cells, after they were labeled with the DNA metabolic label. In addition, expression of the cell membrane-bound TNF family receptors involved in apoptosis mediated by immune effector cells, such as TNFR1, TNFR2, Fas and LT-βR, was investigated using flow cytometry. BrdU labeling of the cell nuclear DNA was found to have no significant effect on the cellular functions tested.

### Improvement of MCA by Utilization of BrdU-Labeled Target Cells

By means of cytotoxicity assays and experimental approaches selective for evaluation of cell apoptosis or necrosis, the inventor has demonstrated that freshly isolated non-activated human peripheral blood NK cells can efficiently mediate apoptotic, but not necrotic, mechanism of killing against a large variety of solid tissue-derived tumor cell lines (6). The inventor has further determined that the anticancer apoptotic activity of NK cells likewise could be detected, using OMCA (14). In addition, he has found that OMCA could detect both apoptotic and necrotic mechanisms of killing cancer cells mediated by IL-2-activated NK (A-NK) cells. In these experiments, however, OMCA showed significant levels of inconsistency, low sensitivity, and low reproducibility.

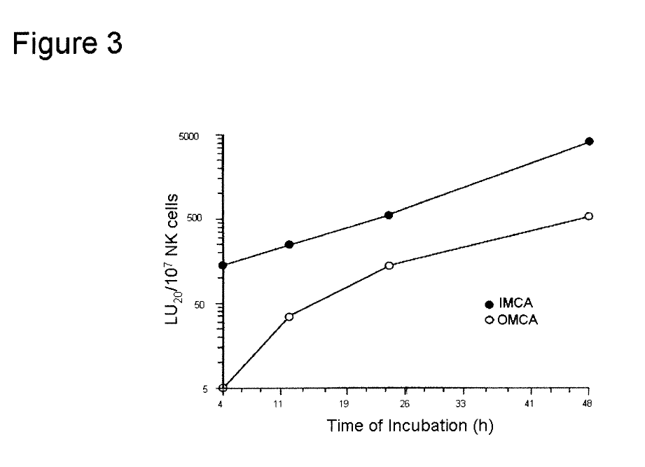

Accordingly, the inventor tested whether utilization of BrdU-pre-labeled target cells, pursuant to the present invention, could help to overcome these weaknesses and improve the microcytotoxicity assay significantly. More specifically, he compared an assay performed with unlabeled target cells and, in accordance with the invention, an assay with BrdU-labeled target cells. In both cases, visual and computerized analyses of target cells revealed that freshly isolated, non-activated PBMNL of normal donors killed cancer target cells, with similar kinetics and efficiency (see Table 1 and FIG. 3). The proportions of killed target cells and LU₂₀/10⁷ NK cells were significantly higher, however, and data obtained by visual and computerized analyses were much more similar with MCA performed with BrdU-labeled target cells, according to the present invention, than that with unlabeled target cells. The inventor also observed that, in MCA performed with BrdU-labeled target cells, the proportion of unlabeled nuclei of target cells was increased in experimental wells, in comparison to control wells.

These findings indicate that a significant proportion of apoptotic target cells remained adherent, following co-incubation with immune effector cells. Because they released fragmented DNA, however, their nuclei became poorly labeled and can therefore be easily differentiated from live cells.

### Application of IMCA and Pre-Labeled Cell Populations

IMCA displays important abilities to detect the major types of cell death, necrosis and apoptosis, and to measure cytotoxic activity of a cytotoxic agent, such as UV radiation, NK cells and CTLs. These abilities underscore the prospect of using the methodology of the invention to measure cytotoxicity that is induced by ionized radiation, cytotoxic drugs, or immune effector cells. In addition, IMCA has a unique capability to detect cancer-related suppression of NK cell-mediated cytotoxicity in cancer patients. Accordingly, IMCA can become a laboratory assay of choice, employed to measure killing of target cells in a variety of experimental, routine pharmacological, and clinical studies, exemplified by the following:

1. monitoring killing of target cells;
2. evaluating the effectiveness of a cytotoxic agent;
3. screening and evaluation of novel anticancer cytotoxic drugs;
4. selection of optimal anticancer cytotoxic therapy;
5. determination of abnormalities of NK cell-mediated killing of cancer cells in cancer patients as a prognostic surrogate marker;
6. testing in vitro cytokine stimulation of NK cell-mediated killing of cancer cells, to predict potential efficacy of cytokine therapy and immunotherapy in cancer patients;
7. monitoring in vivo changes of NK cell-mediated killing of cancer cells during cytokine therapy of cancer patients, as a possible surrogate marker for efficiency of this therapy; and
8. monitoring generation or augmentation of CTL responses after specific, vaccine-based anticancer therapies, to determine effectiveness of the immunization.

Additionally, BrdU-labeling of populations of proliferating cells could be applied, in keeping with the present invention, for testing their in vivo migration. This could be performed, for example, by immunohistochemical staining and analyses of tissue sections following adoptive transfer of in vitro-expanded and BrdU-labeled tumor cells, NK cells, CTLs, or dendritic cells.

### Advantages of IMCA Over OMCA

Introduction of pre-labeling of target cells significantly increases accuracy of target cell counting. By virtue of the resultant selective and distinct staining, according to the invention, cell nuclei are more uniform, better defined, and better segregated objects for counting than whole cells. Pre-labeling of target cells before cell-mediated cytotoxicity significantly increased selectivity of their counting, too, by exclusion of counting remaining immune effector cells as target cells. Labeling of DNA in cell nuclei significantly increased sensitivity of the assay, by enabling identification and differential analysis or exclusion of apoptotic cells. In comparison to other cytotoxicity assays, moreover, IMCA is less complex and easier to perform, is less expensive, using minute quantities of reagents, is more practical, providing semi-automated testing of large numbers of samples, and is safer, using nontoxic or non-radioactive reagents.

### CITED LITERATURE

1. Berke, G. 1994. The binding and lysis of target cells by cytotoxic lymphocytes: Molecular and cellular aspects. Annu. Rev. Immunol. 12: 735-773.
2. Brunner, K. T., J. Mauel, J. C. Cerottini, and B. Chapnis. 1968. Quantitative assay of the lytic action of immune lymphoid cells on ⁵¹Cr-labeled allogeneic target cells in vitro: inhibition by isoantibody and by drugs. Immunol. 14: 181-196.
3. Brunner, K. T, J. Mauel, H. Rudolf, and B. Chapnis. 1970. Studies of allograft immunity in mice. I. Induction, development and in vivo assay of cellular immunity. Immunol. 18: 501-515.
4. Takasugi, M., and E. Klein. 1970. A microassay for cell-mediated immunity. Transplantation. 9: 219-227.
5. Kornblith, P. L., B. H. Smith, and L. A. Leonard. 1981. Response of cultured human brain tumors to nitrosoureas: Correlation with clinical data. Cancer. 47: 255-265.
6. Vujanovic, N. L., S. Nagashima, R. B. Herberman, and T. L. Whiteside. 1996. Non-secretory apoptotic killing by human natural killer cells. J. Immunol. 157: 1117-1126.
7. Monks, A., D. Scudiero, P. Skehan et al. 1991. Feasibility of a high-flux anticancer drug screening using devise panel of cultured human tumor cell lines. J. Natl. Cancer Inst. 83: 757-766.
8. Andreotti, P. E., I. A. Cree, C. M. Kurbacher, et al. 1995. Chemosensitivity testing of human tumors using a microplate adenosine triphosphate luminescence assay: clinical correlation for cisplatin resistance of ovarian carcinoma. Cancer Res. 55: 5276-5282.
9. Csoka, K. R. Larsson, B. Tholander, E. Gerdin, M. de la Torre, and P. Nygren. 1994. Cytotoxic drug sensitivity testing of tumor cells from patients with ovarian carcinoma using the fluorometric microculture cytotoxicity assay (FMCA). Gynecol. Oncol. 54: 163-170.
10. Gratzner, H. G. 1982. Monoclonal antibody to 5-bromo- and 5-iodeoxy-uridine: A new reagent for detection of DNA replication. Science. 218: 474-475.
11. Magaud, J.-P., I. Sargent, and D. Y. Mason. 1988. Detection of human white cell proliferative responses by immunoenzymatic measurement of bromodeoxyuridine uptake. J. Immunol. Methods. 106: 95-100.
12. Wilson, G. D., N. J. McNally, E. Dunphy, H. Karcher, and R. Pfragner. 1985. The labeling index of human and mouse tumors assessed by bromodeoxyuridine staining in vitro and in vivo and flow cytometry. Cytometry. 6: 641-647.
13. Vakkila, J., R. A. DeMarco, and M. T. Lotze. 2004. Imaging analysis of STAT1 and NF-kB translocation in dendritic cells at the single cell level. J. Immunol. Methods 294:123-134.
14. Wahlberg B. J., D. R. Burholt, P. Kornblith, T. Richards, A. Bruffsky, R. B. Herberman, N. L. Vujanovic. 2001. Measurement of NK activity by the microcytotoxicity assay (MCA): A new application for an old assay. J. Immunol. Meth. 253:69-81.

TABLE 1
Comparison of OMCA and IMCA.¹
% Cytotoxicity (LU₂₀/10⁷ NK cells)

| | OMCA | | | IMCA | |
|---|---|---|---|---|---|
| Experiment | E:T | Visual | Computer | Visual | Computer |
| 1 | 200:1 | 50 | 33 | 57 | 48 |
| | 100:1 | 39 | 17 | 49 | 46 |
| | 50:1 | 16 | 0 | 21 | 15 |
| | 25:1 | 3 | 0 | 0 | 0 |
| | | (482) | (132) | (654) | (559) |
| 2 | 200:1 | 14 | 8 | 31 | 34 |
| | 100:1 | 10 | 0 | 21 | 19 |
| | 50:1 | 9 | 1 | 16 | 11 |
| | 25:1 | 9 | 6 | 5 | 4 |
| | | (104) | (39) | (350) | (339) |

¹BT-20 target cells were seeded in microwells of Tarasaki miniplates in the absence or presence of BrdU and incubated for 24 hours. The unlabeled and BrdU-labeled adherent target cells were then exposed to normal donor PBMNL in 4 different E:T ratios for 24 h. Following this cytotoxic treatment of target cells, OMCA and IMCA were respectively completed. The analyses were performed by visual or computerized counting of remaining target cells. The assays were done in six replicates. The results are mean percentages of cytotoxicity. In parentheses are LU₂₀/10⁷ NK cells. The major differences between the results obtained in IMCA and OMCA, and between the data obtained by visual and computerized analyses in MCA were significant (p < 0.05).

What is claimed is:

1. A microcytotoxicity assay comprised of: (A) providing a culture of target cells adhered to a surface, wherein said target cells are exposed to a labeling agent that labels nuclei of said target cells; then (B) treating said target cells with a putative cytotoxic agent; and thereafter (C) assessing the number of cells that remain adhered to said surface.

2. A microcytotoxicity assay according to claim 1, wherein said target cells are tumor cells.

3. A microcytotoxicity assay according to claim 2, wherein said putative cytotoxic agent is selected from the group consisting of immune cells, a chemical compound, UV radiation, and X-ray radiation.

4. A microcytotoxicity assay according to claim 3, wherein said immune cells are selected from the group consisting of natural killer cells, cytotoxic T lymphocytes, natural killer T cells, dendritic cells, and combinations thereof.

5. A microcytotoxicity assay according to claim 1, wherein said labeling agent is 5-bromo-2′-deoxyuridine.

6. A microcytotoxicity assay according to claim 1, wherein said labeling agent is selected from the group consisting of 4′,6-diamidino-2-phenylindole (DAPI), a Hoechst fluorescent stain, and a cell-permeante cyanine nucleic acid stain.

7. A microcytotoxitiy assay according to claim 1, wherein said surface is a surface of a microwell into which said target cells are plated.

8. A microcytotoxicity assay according to claim 7, wherein said target cells are distributed among a plurality of microwells.

9. A microcytotoxicity assay according to claim 1, wherein said target cells are normal cells.

10. A microcytotoxicity assay according to claim 9, wherein said putative cytotoxic agent is selected from the group consisting of a chemical compound, UV radiation, and X-ray radiation.

11. A combination comprised of (i) a suspension of cells that can form an adherent layer, wherein said cells are labeled by an agent that labels nuclei thereof, and (ii) instructions for the use of said cells to effect a microcytotoxicity assay according to claim 1.

12. A combination according to claim 11, further comprising (iii) a microcytotoxicity plate, wherein said instructions relate said use to said plate for effecting said microcytotoxicity assay.
